(19) United States
(12) Patent Application Publication
(10) Pub. No.: US 2010/0145232 A1
(43) Pub. Date: Jun. 10, 2010

(54) METHODS AND APPARATUSES FOR CORRECTING SPORT POSTURES CAPTURED BY A DIGITAL IMAGE PROCESSING APPARATUS

(75) Inventors: Soon-geun Jang, Suwon-si (KR); Ung-sik Kim, Suwon-si (KR)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: Samsung Digital Imaging Co., Ltd., Suwon-si (KR)

(21) Appl. No.: 12/625,851

(22) Filed: Nov. 25, 2009

(30) Foreign Application Priority Data
Dec. 4, 2008 (KR) 10-2008-0122602

(51) Int. Cl. A61B 5/103; G06K 9/00

(52) U.S. Cl. 600/587; 382/128

(57) ABSTRACT

A sport posture correcting apparatus including a digital image processing apparatus configured to generate a series of image frames. The sport posture correcting apparatus may include a digital signal processing unit configured to set a region in one of the series of generated image frames. The digital signal processing unit may be configured to trace the set region in the series of generated frames to generate a tracing result. The digital signal processing unit may be configured to display the tracing result.

### CROSS-REFERENCE TO RELATED PATENT APPLICATIONS

This application claims the benefit of Korean Patent Application No. 10-2008-0122602, filed on Dec. 4, 2008 in the Korean Intellectual Property Office, the entire contents of which is incorporated herein by reference.

### BACKGROUND OF THE INVENTION

1. Field of the Invention

The present invention relates to digital image processing apparatuses and methods thereof for correcting a sport person's postures.

2. Description of the Related Art

When an expert in a particular sport wants to correct postures of a trainee, the expert generally may refer to moving pictures of 30 frames per second (FPS) which is the maximum many camcorders are capable of recording. In some sports centers, when the trainee wears markers and makes postures, the experts may correct the postures of the trainee by tracing the markers using a computer. However, the above system is expensive and requires well-defined equipment (an image-taking room, expensive camera equipment, and a computer for numerical analysis), and it is difficult to distribute the above system widely.

### SUMMARY OF THE INVENTION

The present invention provides methods and apparatuses for correcting a sport person's postures.

A sport posture correcting apparatus may include a digital image processing apparatus configured to generate a series of image frames. The sport posture correcting apparatus may include a digital signal processing unit configured to set a region in one of the series of generated image frames. The digital signal processing unit may be configured to trace the set region in the series of generated frames to generate a tracing result. The digital signal processing unit may be configured to display the tracing result.

The digital signal processing unit may be configured to simultaneously display the tracing result and a reference trace that is stored in advance in the sport posture correcting apparatus.

The digital signal processing may include a region setting unit configured to set a region in an image frame from the series of image frames; and may include a tracing unit configured to trace the set region in the series of image frames to generate a tracing result; and may include a tracing result displayer configured to output the tracing result as a signal that can be displayed.

The tracing unit may be configured to: set the location of the set region in a current image frame from the series of image based on the set region in a previous image frame from the series of images frames; and may be configured to change the location of the set region in the current image frame along a first coordinate to where a calculated similarity is highest along the first coordinate; and may be configured to change the location of the region along a second coordinate of the current frame to where a calculated similarity is highest along the second coordinate; and may be configured to confirm that the set region in the current frame is a tracing region from the set region of the previous frame based on a result of the similarity calculation.

The tracing result displayer may be configured to output tracing results with respect to the series of image frames as signals that can be displayed.

A sport posture correcting apparatus may include a digital image processing apparatus configured to generate a series of image frames of a first user and a second user. The sport posture correcting apparatus may include a digital signal processing unit configured to set regions in the series of image frames showing postures of the first user and postures of the second user; and may be configured to trace the set region in the series of generated frames to generate a tracing result for the first user and a tracing result for the second user; and may be configured to simultaneously display the tracing results for the first user and the second user.

The digital signal processing unit may include a region setting unit configured to set a region in an image frame from the series of image frames of the first user and of the second user.

The digital signal processing unit may include a tracing unit configured to trace the set region in the series of image frames of the first user and to trace the set region of the second user to generate a tracing result for the first user and for the second user.

The digital signal processing unit may include a tracing result displayer configured to output the tracing result of the first user and of the second user as a signal that can be displayed.

The tracing unit may be configured to set the location of the set region in a current image frame based on the set region in a previous image frame from the series of images frames for the first user and from the second user; and may be configured to change the location of the set region in the current image frame along a first coordinate to where a calculated similarity is highest along the first coordinate; and may be configured to change the location of the set region along a second coordinate of the current frame to where a calculated similarity is highest along the second coordinate; and may be configured to confirm that the set region in the current frame is a tracing region from the set region of the previous frame based on a result of the similarity calculation.

The tracing result displayer may be configured to output tracing results with respect to the series of image frames of the first user and the second user as signals that can be displayed.

A method of correcting sport postures may include generating a series of image frames; and may include setting a region in an image frame from among the series of image frames; and may include tracing the set region in other image frames of the series of image frames to generate a result; and may include displaying the result of the tracing of the set region in the series of image frames.

Tracing the set region may include setting the location of the set region in a current image frame based on the set region in a previous image frame from the series of images frames; and changing the location of the set region in the current image frame along a first coordinate to where a calculated similarity is highest along the first coordinate; and changing the location of the region along a second coordinate of the current frame to where a calculated similarity is highest along the second coordinate; and confirming that the set region in the current frame is a tracing region from the set region of the previous frame based on a result of the similarity calculation.

Displaying the result of the tracing may include displaying the result of the tracing with respect to entire image frames.

Displaying the result of the tracing may include displaying the result of the tracing of the set region in the series of image frames simultaneously with reference traces stored in advance in the digital image processing apparatus.

A method of correcting sport postures may include generating a series of image frames showing sport postures of a first user and a series of image frames showing sport postures of a second user; and may include setting a region in an image frame from among the series of image frames showing the first user and from among the series of image frames showing the second user; and may include tracing the set region in other image frames of the series of image frames showing the first user to generate a first result and tracing the set region in other image frames of the series of image frames showing the second user to generate a second result; and may include displaying the first and second result of the tracing.

Tracing the set region may include setting the location of the set region in a current image frame based on the set region in a previous image frame from the series of images frames; and may include changing the location of the set region in the current image frame along a first coordinate to where a calculated similarity is highest along the first coordinate; and may include changing the location of the region along a second coordinate of the current frame to where a calculated similarity is highest along the second coordinate; and may include confirming that the set region in the current frame is a tracing region from the set region of the previous frame based on a result of the similarity calculation.

Displaying may include displaying simultaneously the first and second result of the tracing.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of an example of a sport posture correcting apparatus in a digital image processing apparatus according to an embodiment of the present invention;

FIG. 2 is a detailed block diagram of the digital signal processor shown in FIG. 1;

FIG. 3 is a detailed block diagram of an example of a tracing unit shown in FIG. 2;

FIG. 4 is a diagram showing examples of image frames that are captured successively in FIG. 1 and a region tracing result;

FIG. 5 is a diagram for explaining region tracing in the apparatus of FIG. 1;

FIG. 6 is a diagram showing an exemplary display of the region tracing result in the apparatus of FIG. 1;

FIG. 7 is a flowchart illustrating an example of a process of correcting postures captured by a digital image processing apparatus according to an embodiment of the present invention;

FIG. 8 is a flowchart illustrating an example of a process of region tracing; and

FIG. 9 is a flowchart illustrating an example of a process of correcting postures captured by a digital image processing apparatus according to another embodiment of the present invention.

### DETAILED DESCRIPTION OF THE INVENTION

Hereinafter, the present invention will be described with reference to accompanying drawings.

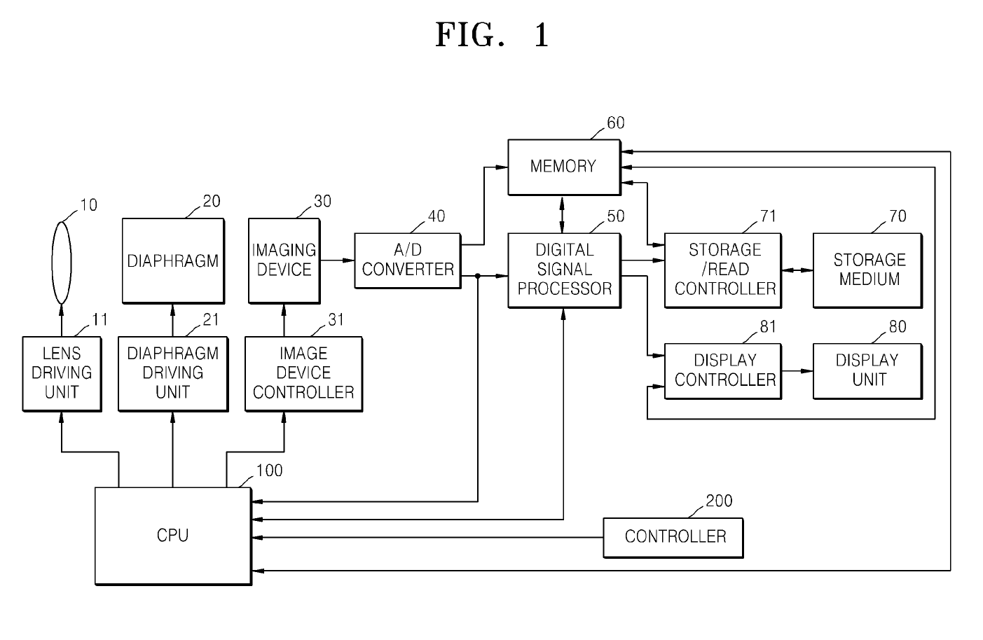

FIG. 1 is a block diagram showing an example of a sport posture correcting apparatus in a digital image processing apparatus according to an embodiment of the present invention. In particular, FIG. 1 schematically shows a digital photographing apparatus as an example of the digital image processing apparatus. The present invention is not limited to the digital photographing apparatus shown in FIG. 1, and the present invention may be applied to image processing apparatuses such as personal digital assistants (PDAs) and personal multimedia players (PMPs).

All operations of the digital photographing apparatus may be controlled by a central processing unit (CPU) 100. The digital photographing apparatus may include a controller 200 including keys for generating electric signals input from a user. The electric signals generated by the controller 200 may be transferred to the CPU 100 so that the CPU 100 controls the digital photographing apparatus according to the electric signals.

In a photographing mode, when the electric signal from the user is applied to the CPU 100, the CPU 100 may control a lens driving unit 11, a diaphragm driving unit 21, and an imaging device controller 31 according to the electric signal, and accordingly, a position of a lens 10, an opening degree of the diaphragm 20, and the sensitivity of the imaging device 30 may be controlled. The imaging device 30 generates data from input light, and an analog/digital (A/D) converter 40 converts analog data output from the imaging device 30 to digital data. The A/D converter 40 may not be necessary according to characteristics of the imaging device 30.

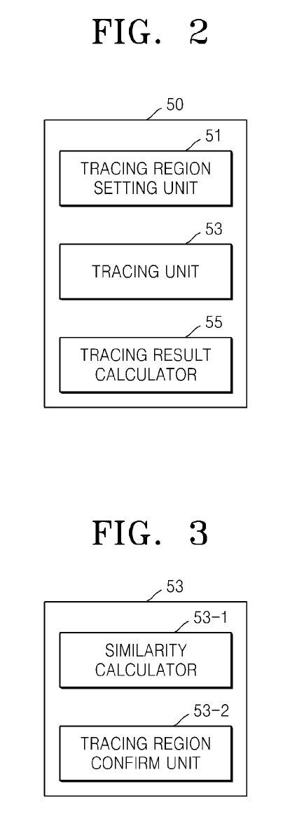

The data output from the imaging device 30 may be input into a digital signal processor 50 via a memory 60, or may be input into the digital signal processor 50 without passing through the memory 60, or may be input into the CPU 100. The memory 60 may include a read only memory (ROM) and/or a random access memory (RAM). The digital signal processor 50 may perform digital signal processing such as gamma compensation and white balance adjustment. In addition, as shown in FIG. 2, the digital signal processor 50 may include a tracing region setting unit 51, a tracing unit 53, and a tracing result calculator 55. The tracing region setting unit 51, the tracing unit 53, and the tracing result calculator 55 may or may not be included in the digital signal processor 50. The tracing region setting unit 51 may set a certain region from an image frame, the tracing unit 53 may trace the set region in other image frames, and the tracing result calculator 55 may output the region tracing result as a signal that can be displayed. Operations of the tracing region setting unit 51, the tracing unit 53, and the tracing result calculator 55 will be described later.

Referring back to FIG. 1, images of the data output from the digital signal processor 50 may be transferred to a display controller 81 directly or through the memory 60. The display controller 81 may control a display unit 80 to display moving pictures on the display unit 80. In addition, the data output from the digital signal processor 50 may be input into a storage/read controller 71 directly or through the memory 60, and the storage/read controller 71 may store the image data in a storage medium 70 automatically or according to a signal from the user. The storage/read controller 71 may read data from the moving picture file stored in the storage medium 70, and may input the data into the display controller 81 through the memory 60 or via other paths to display the moving pictures on the display unit 80. The storage medium 70 may be detachable or fixedly mounted in the digital photographing apparatus.

Hereinafter, functions of the tracing region setting unit 51, the tracing unit 53, and the tracing result calculator 55 will be described with reference to the accompanying drawings.

FIG. 4 (a) shows a series of image frames of sport postures, for example, swing postures that are photographed by the digital photographing apparatus of FIG. 1. In the present embodiment, golf swing postures are represented as an example of the sport posture, however, the present invention is not limited thereto and may be applied to various sports, for example, baseball, swimming, etc. The tracing region setting unit 51 may set a certain region that will be traced in a first image frame from among the image frames shown in FIG. 4 (a). The tracing region setting unit 51 may set regions of hands, shoulders, waist, and legs as the regions that will be traced. The tracing region setting unit 51 may set regions based on user selection of an area of the image or may set regions based on user selection of a an area of the body. In FIG. 4 (a), when the tracing region setting unit 51 selects the region of the hands as a set region 400, the tracing result of the hands is displayed as shown in FIG. 4 (b).

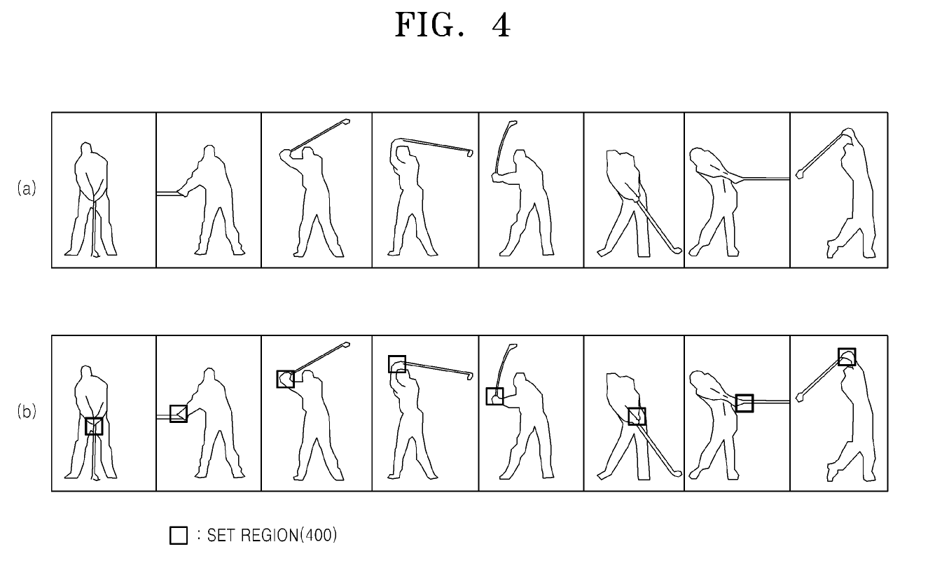

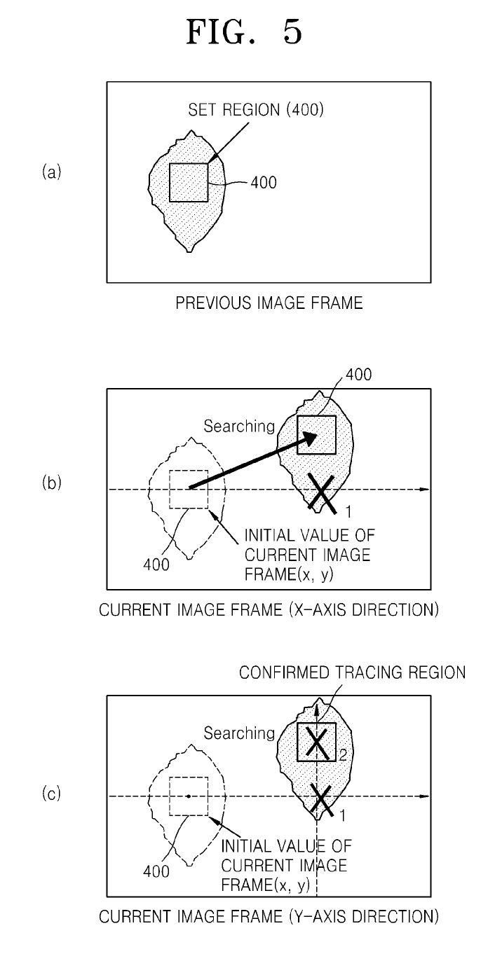

The tracing unit 53 may trace the set region 400 in images of the series image frames. FIG. 3 shows the tracing unit 53 that traces the set region 400, and the tracing unit 53 includes a similarity calculator 53-1 and a tracing region confirmation unit 53-2. Detailed operations of the tracing unit 53 will be described with reference to FIGS. 3 and 5.

The similarity calculator 53-1 may calculate a position for the set region 400 in a current image frame by first setting an initial value (x, y) for the set region 400 in the current image frame as the same location as the set region 400 in a previous image frame as shown in FIG. 5 (a).

The similarity calculator 53-1 may then calculate an x-axis position in the current frame, as shown in FIG. 5 (b). The x-axis position may be calculated while fixing a location of the set region 400 in a y-axis position in the current frame and changing a location of the set region 400 in the x-axis direction. The object that is to be traced may not be a point, but an area having a constant color saturation or a brightness value with a constant pattern, and thus, a point represented as X₁ in FIG. 5 (b) is detected as the location where the similarity in the x-axis direction is calculated by the similarity calculator 53-1 as being the highest.

The similarity calculation may be performed between the set region of the previous image and the current set region of the current image using a sum of absolute differences (SAD, which is the sum of absolute values between differences of two images), a sum of squared differences (SSD), a zero-mean normalized cross-correlation (ZNCC), or mutual information (using a probability of a two-dimensional histogram).

When the x-axis position is calculated, the similarity calculator 53-1 may calculate the y-axis direction while fixing the x-axis position. In FIG. 5 (c), the point represented as X₂ is detected to be the location where the similarity in the y-axis direction is calculated by the similarity calculator 53-1 as being the highest.

The tracing region confirmation unit 53-2 confirms that the set region 400 in the current frame is a good match for the set region from the previous frame based on the result of a calculation by the similarity calculator 53-1 measuring the difference between the set region 400 of the current frame and the set region of the previous frame. The confirmation unit 53-2 may start the process of finding the set region 400 of the current frame over again if the calculation by the similarity calculator 53-1 indicates that the set region 400 of the current frame may not be the set region from the previous frame.

The tracing result calculator 55 may simultaneously display traces of the tracing regions of the set region 400, which are determined by the tracing unit 53 from the series of image frames. The tracing result calculator 55 may display the traces of an expert (reference trace) stored in the memory 60 in advance and traces of the tracing results simultaneously.

In another embodiment, sport postures of a trainee (for example, a first user) are successively photographed, and sport postures of the expert (for example, a second user) are successively photographed, and then, the same set regions are traced in both the trainee and expert posture images. After that, the tracing results are simultaneously displayed to compare the sport postures of the trainee with those of the expert.

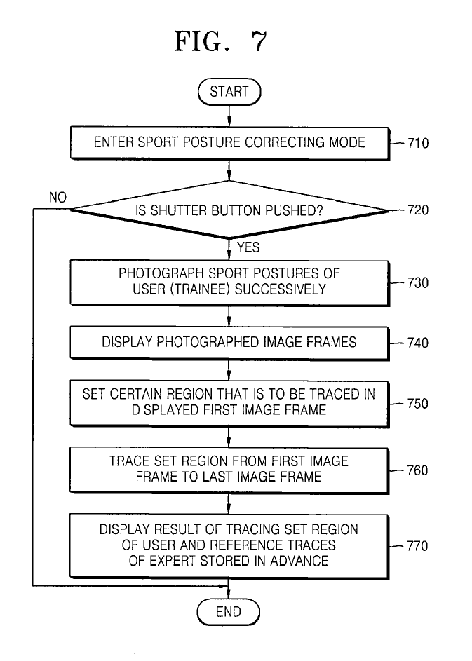

An example of a method of correcting postures will be described with reference to FIGS. 7 through 9. The posture correcting method of the present embodiment may be performed in the digital photographing apparatus shown in FIGS. 1 through 3, and the method of the operations may be performed in the digital signal processor 50 with the help of peripheral elements.

First, an example of a method of correcting sport postures captured by the digital image processing apparatus according to an embodiment of the present invention will be described with reference to FIG. 7 as follows.

The digital signal processor 50 that receives a menu selection signal provided by the digital image processing apparatus, for example, the digital photographing apparatus, enters a sport posture correction mode in the digital image processing apparatus (S710).

When the digital image processing apparatus enters the sport posture correction mode, the digital signal processor 50 determines whether a shutter button is pressed by a user, for example, the trainee (S720). When the shutter button is pressed, the digital signal processor 50 successively takes a series of images of the postures of the user (S730). In FIG. 4 (a), golf swing postures that are photographed successively are shown.

After taking the images, the digital signal processor 50 may display one or more image frames that are captured successively on the display unit 80 (S740). The digital signal processor 50 receives a signal for setting a certain region that will be traced from the first image frame to the last image frame from among the displayed image frames (S750). In embodiments, not every image frame is traced. In FIG. 4 (b), the region 400 that is to be traced is set in the first image frame. In embodiments, the region 400 can be selected from any image frame. In the present invention, golf swing postures are represented, however, the present invention is not limited to the above example, but may be applied to various sports, such as baseball, swimming, etc. The digital signal processor 50 may set the region including hands, shoulders, waist, or legs as the region that is to be traced.

When the region that is to be traced is set, the digital signal processor 50 traces the set region 400 from the first image frame to the last image frame (S760). In embodiments, not every image is traced. In embodiments, the set region 400 may be selected in the middle of the series of images and the set region 400 will be traced both backward and forward in the series of images.

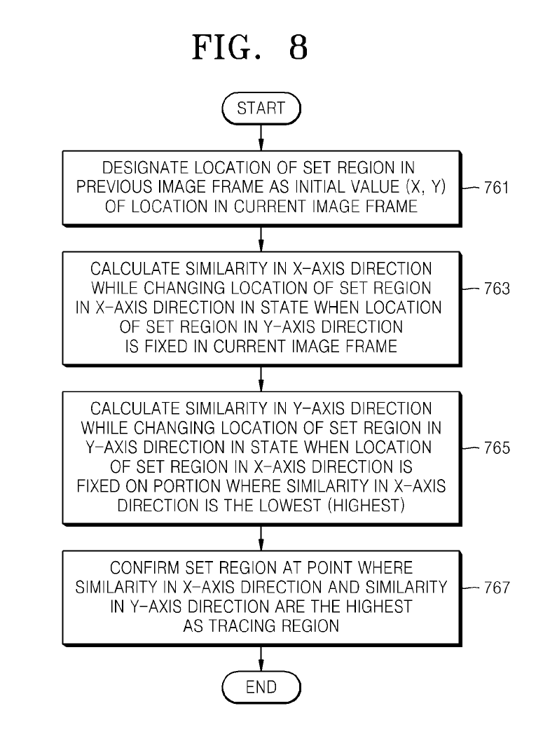

FIG. 8 shows an example of a process of tracing the set region 400 by using the digital signal processor 50.

The digital signal processor 50 designates the location (x, y) of the set region 400 in the previous frame as an initial value of a location in the current frame as shown in FIG. 5 (a) (S761).

Then, the digital signal processor 50 calculates the similarity in the x-axis direction in the current frame as shown in FIG. 5 (b). The similarity in the x-axis direction is calculated while changing the location of the set region 400 in the x-axis direction in a state when the location of the set region 400 in the y-axis direction is fixed (S763). In this case, since the object that is to be traced may not be a point, but an area having a constant color saturation or a brightness value with a constant pattern, and thus, the portion represented as X₁ in FIG. 5 (b) is detected as the location where the similarity is the highest in 5 (c). The similarity may be calculated by using the above described methods.

When the similarity in the x-axis direction is calculated, the digital signal processor 50 calculates the similarity in the y-axis direction while changing the location of the set region in the y-axis direction in a state when the set region 400 is fixed in the x-axis direction (S765). Referring to FIG. 5 (c), the portion represented as X₂ is detected as the location where the similarity in the y-axis direction is the highest.

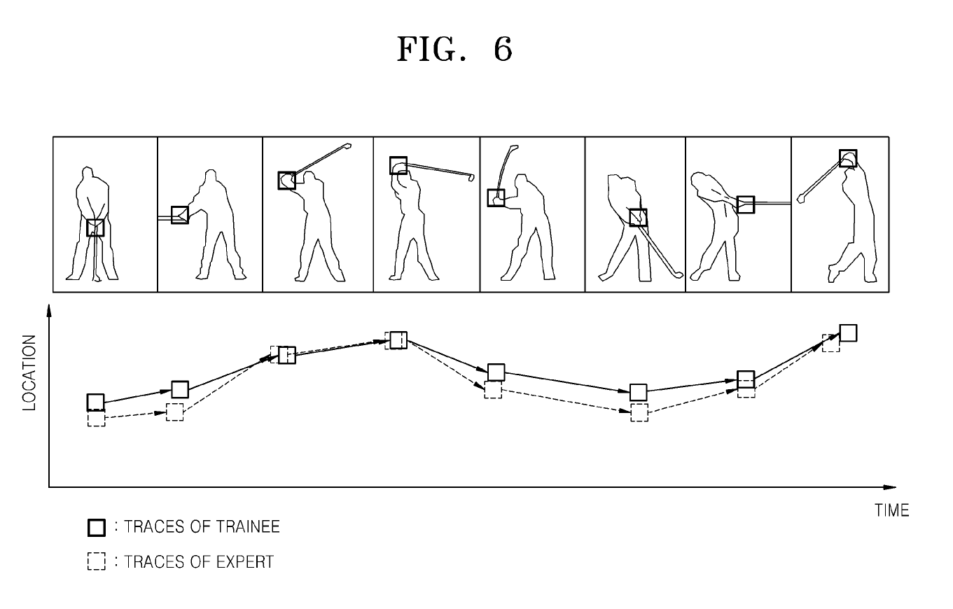

After that, the digital signal processor 50 confirms that the set region 400 is the region of the current image most similar to the set region of the previous image according to the similarity calculation in the x-axis direction and the y-axis direction (S767). As described above, when the tracing of the set region 400 is finished in all the images of the series of images, the digital signal processor 50 simultaneously displays the traces of the confirmed tracing regions (S770). At this time, the digital signal processor 50 may display the traces of the expert (reference traces) stored in the memory 60 in advance and the traces of the calculated tracing results simultaneously. FIG. 6 shows the traces of the set regions for all of the image frames, and accordingly, the postures of the trainee, which are to be corrected, may be compared with the postures of the expert.

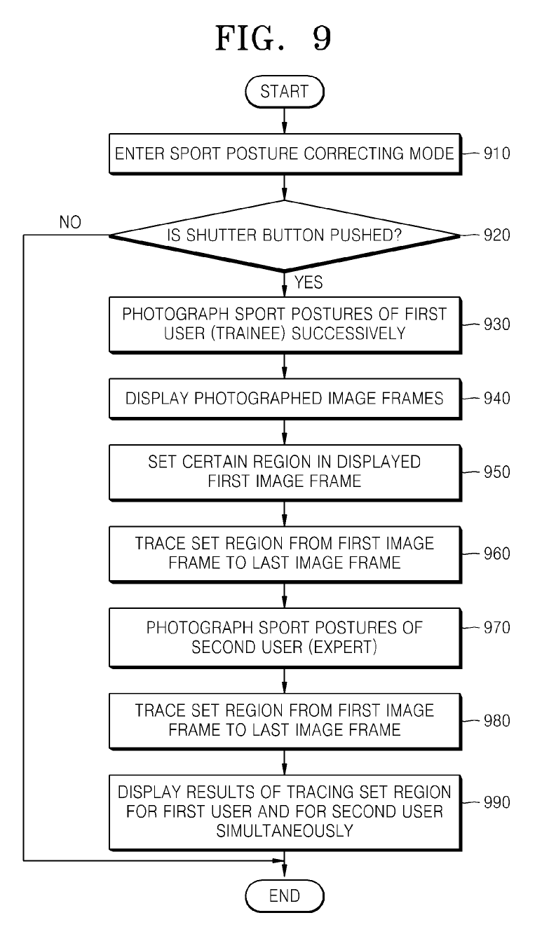

Next, an example of a method of correcting sport postures captured by the digital image processing apparatus according to another embodiment of the present invention will be described with reference to FIG. 9.

The digital signal processor 50 that receives a menu selection signal provided by the digital image processing apparatus, for example, the digital photographing apparatus, enters a sport posture correction mode in the digital image processing apparatus (S910).

When the digital image processing apparatus enters the sport posture correction mode, the digital signal processor 50 determines whether a shutter button is pressed or not (S920).

When the shutter button is pushed, the digital signal processor 50 may successively capture a series of images of sport postures of a first user, for example, a trainee (S930).

After the successive photographing operation is finished, the digital signal processor 50 displays one or more image frames obtained by the photographing operation (S940).

The digital signal processor 50 may receive a signal for setting a certain region that will be traced from the first image frame to the last image frame from among the displayed image frames (S950). In (b) of FIG. 4, a certain region 400 that is to be traced is set in the first image frame.

When the region that is to be traced is set, the digital signal processor 50 traces the set region 400 from the first image frame to the last image frame (S960). Processes of tracing the set regions 400 are described with reference to FIG. 8 in the above embodiment, and thus, detailed descriptions thereof will be omitted here.

When the tracing of the set regions 400 in the image frames showing the postures of the first user, sport postures of a second user (an expert) may be photographed successively in a series of images(S970). In embodiments, the series of images of the second user may be taken before the series of images of the first user. In embodiments, the tracing of the first series of images is done after the series of images of the second user are taken.

After the photographing process, the digital signal processor 50 may trace the set regions 400 from the first image frame to the last image frame (S980).

As described above, when the tracing of the set regions 400 for all of the image frames is finished, the digital signal processor 50 displays the traces of the set regions of the first and second users at the same time (S990). FIG. 6 shows the traces of the set regions for all of the image frames, and accordingly, the postures of the trainee, which are to be corrected, may be compared with the postures of the expert.

According to the present invention, a certain region in images showing a sport person's postures that are photographed successively may be traced to correct the postures, and accordingly, the postures may be corrected easily without using a conventional complex system for correcting postures.

For convenience, in the description above, the functionality described has been divided into a number of units; however, the number of units may vary and the functionality described above may be differently divided among the units, or the functionality described above may be implemented without units.

The various illustrative units described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

Also, functional programs, codes, and code segments for accomplishing the present invention can be construed by programmers skilled in the art to which the present invention pertains.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A sport posture correcting apparatus comprising,
a digital signal processing unit configured to generate a series of image frames, configured to set a region in one of the series of generated image frames, and to trace the set region in the series of generated frames to generate a tracing result, and configured to display the tracing result.

2. The sport posture correcting apparatus of claim 1, wherein the digital signal processing unit is configured to simultaneously display the tracing result and a reference trace that is stored in advance in the sport posture correcting apparatus.

3. The sport posture correcting apparatus of claim 1, wherein the digital signal processing unit comprises:
a region setting unit configured to set a region in an image frame from the series of image frames;
a tracing unit configured to trace the set region in the series of image frames to generate a tracing result; and
a tracing result displayer configured to output the tracing result as a signal that can be displayed.

4. The sport posture correcting apparatus of claim 3, wherein the tracing unit is configured to:
set the location of the set region in a current image frame from the series of image frames based on the set region in a previous image frame from the series of images frames;
change the location of the set region in the current image frame along a first coordinate to where a calculated similarity is highest along the first coordinate;
change the location of the region along a second coordinate of the current frame to where a calculated similarity is highest along the second coordinate;
and confirm that the set region in the current frame is a tracing region from the set region of the previous frame based on a result of the similarity calculation.

5. The sport posture correcting apparatus of claim 3, wherein the tracing result displayer is configured to output tracing results with respect to the series of image frames as signals that can be displayed.

6. A sport posture correcting apparatus comprising:
a digital signal processing unit configured to generate a series of image frames of a first user and a second user, configured to set regions in the series of image frames showing postures of the first user and postures of the second user, and configured to trace the set region in the series of generated frames to generate a tracing result for the first user and a tracing result for the second user and configured to simultaneously display the tracing results for the first user and the second user.

7. The sport posture correcting apparatus of claim 6, wherein the digital signal processing unit comprises:
a region setting unit configured to set a region in an image frame from the series of image frames of the first user and of the second user;
a tracing unit configured to trace the set region in the series of image frames of the first user and to trace the set region of the second user to generate a tracing result for the first user and for the second user; and
a tracing result displayer configured to output the tracing result of the first user and of the second user as a signal that can be displayed.

8. The sport posture correcting apparatus of claim 7, wherein the tracing unit is configured to:
set the location of the set region in a current image frame based on the set region in a previous image frame from the series of images frames for the first user and from the second user;
change the location of the set region in the current image frame along a first coordinate to where a calculated similarity is highest along the first coordinate;
change the location of the set region along a second coordinate of the current frame to where a calculated similarity is highest along the second coordinate;
and confirm that the set region in the current frame is a tracing region from the set region of the previous frame based on a result of the similarity calculation.

9. The sport posture correcting apparatus of claim 7, wherein the tracing result displayer is configured to output tracing results with respect to the series of image frames of the first user and the second user as signals that can be displayed.

10. A method of correcting sport postures, the method comprising:
generating a series of image frames;
setting a region in an image frame from among the series of image frames;
tracing the set region in other image frames of the series of image frames to generate a tracing result; and
displaying the result of the tracing of the set region in the series of image frames.

11. The method of claim 10, wherein tracing the set region comprises:
setting the location of the set region in a current image frame based on the set region in a previous image frame from the series of images frames;
changing the location of the set region in the current image frame along a first coordinate to where a calculated similarity is highest along the first coordinate;
changing the location of the region along a second coordinate of the current frame to where a calculated similarity is highest along the second coordinate; and
confirming that the set region in the current frame is a tracing region from the set region of the previous frame based on a result of the similarity calculation.

12. The method of claim 10, wherein displaying the result of the tracing comprises: displaying the result of the tracing with respect to the series of image frames as signals that can be displayed.

13. The method of claim 12, displaying the result of the tracing comprises:
displaying the result of the tracing of the set region in the series of image frames simultaneously with reference traces stored in advance in the digital image processing apparatus.

14. A method of correcting sport postures, the method comprising:
generating a series of image frames showing sport postures of a first user and a series of image frames showing sport postures of a second user;
setting a region in an image frame from among the series of image frames showing the first user and from among the series of image frames showing the second user;
tracing the set region in other image frames of the series of image frames showing the first user to generate a first tracing result and tracing the set region in other image frames of the series of image frames showing the second user to generate a second tracing result; and
displaying the first and second result of the tracing.

15. The method of claim 14, wherein tracing the set region comprises:
setting the location of the set region in a current image frame based on the set region in a previous image frame from the series of images frames for the first user and from the second user;
changing the location of the set region in the current image frame along a first coordinate to where a calculated similarity is highest along the first coordinate;
changing the location of the region along a second coordinate of the current frame to where a calculated similarity is highest along the second coordinate; and
confirming that the set region in the current frame is a tracing region from the set region of the previous frame based on a result of the similarity calculation.

16. The method of claim 14, wherein displaying comprises:
displaying simultaneously the first and second result of the tracing.
